(19) United States
(12) Patent Application Publication
(10) Pub. No.: US 2013/0245354 A1
(43) Pub. Date: Sep. 19, 2013

(54) Automated method and apparatus for the verification of treatment parameters prior to delivery of radiaiton to patient

(71) Applicants: Serguei A. Kriminski, Poughkeepsie, NY (US); Ivan Lysiuk, Daly City, CA (US)

(72) Inventors: Serguei A. Kriminski, Poughkeepsie, NY (US); Ivan Lysiuk, Daly City, CA (US)

(21) Appl. No.: 13/423,257

(22) Filed: Mar. 18, 2012

(51) Int. Cl. A61N 5/00

(52) U.S. Cl. 600/1

(57) ABSTRACT

Prior to delivery of the radiation treatment, the actual treatment parameters need to be verified against the parameters in a treatment planning system. This verification (timeout) is performed manually by radiation technologist(s) as shown in FIG. 2. We propose using an automatic mechanism to perform the timeout. As is shown in FIG. 4, one of the proposed embodiments directly reads the output to the human interface system from the controller of treatment device. Then, the embodiment extracts the necessary information from the output and compares it with the data extracted (in advance) from the treatment planning system. Different embodiments of the automatic timeout are also presented.

### CROSS-REFERENCE TO RELATED APPLICATIONS

Provisional patent application, express mail # EG 711288397 US, sent Apr. 13, 2011

### FEDERALLY SPONSORED RESEARCH

Not applicable

### JOINT RESEARCH AGREEMENT

Not applicable

### SEQUENCE LISTING OR PROGRAM

Not applicable

### BACKGROUND OF INVENTION

1. Field of invention

The present invention is in the field of the radiation therapy; more specifically, it relates to verification of treatment parameters for the radiation treatment devices. Non-exhaustive list of such devices includes teletherapy units, where source of the radiation is outside of the patient's body, and brachytherapy units, when radiation source is near the surface of a patient's body or within the body or bodily cavity.

2. Verification of Treatment Parameters (Timeout) in Radiation Therapy

Workflow of radiation delivery to a patient most often includes the following steps:

The first step is planning of the radiation treatment. This step is performed by a specially trained person, a treatment planner. He, while interacting with a physician, creates a treatment plan. This is done with the aid of a treatment planning system (TPS), usually a computer program. The plan, which is an electronic record of the proposed radiation delivery, inter alia, contains parameters of the treatment to be delivered by the treatment device.

The second step, which commences upon the approval of the plan by the physician, is the transfer of the plan, or part of the plan to a special storage system, commonly referred to as record-and-verify system. This step is usually performed by the treatment planner.

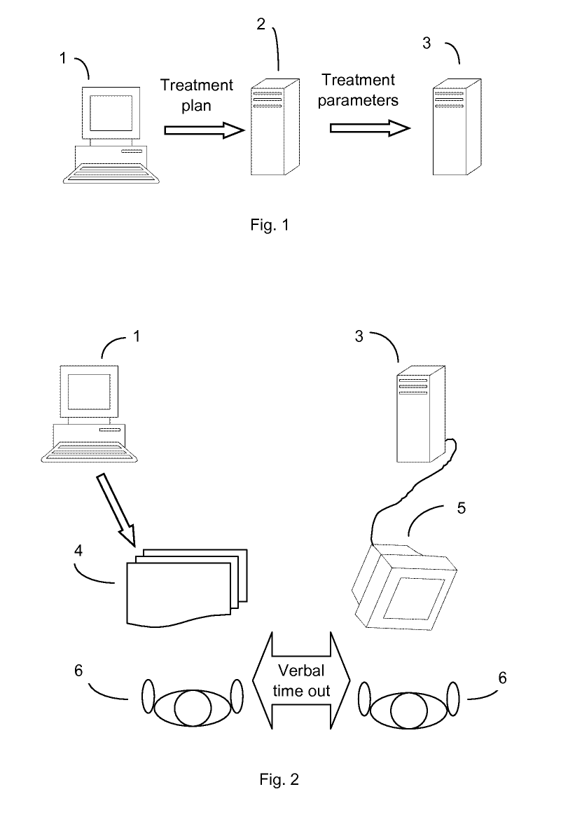

The third step is the transfer of the treatment planning parameters from the record-and-verify system to the radiation delivery device. The flow of the data is indicated in FIG. 1. This step is commonly performed by radiation technologist(s), the personnel specially trained to position the patient and deliver radiation treatment. Sometimes radiation technologists are referred to as radiation therapists or the therapists. The third step is performed immediately prior to the treatment delivery. It should be pointed out that in most cases radiation delivery is repeated many times (called fractions) on, commonly, daily basis. Typical fractionation is 16 to 43 times. Additionally, each fraction may be delivered, depending on the treatment device, in small segments, called fields or arcs.

The fourth step requires some elucidation. Both steps two and step three may involve errors in data transfer. Such errors can be caused by the software, often from different vendors, and hardware, e.g. power failure or poor network connection. Therefore, the fourth step, called a timeout, is the process of verification of the treatment parameters. This step is performed by radiation technologist(s), and is claimed to significantly reduce error rate (B Rasmussen and K Chu, Medical Physics 37, 3450 (2010)) It performed manually via comparison of the parameters from the treatment planning system to the treatment parameters loaded to the radiation delivery device. The treatment planning parameters are viewed on treatment planning system printout, an electronic equivalent of the printout (e.g. in portable document format or PDF), or manual write up from the treatment planning system. Typically, one of the therapists reads the parameters on the screen or monitor of the treatment device and the other therapist confirms them while reading from treatment planning system printout. If the radiation delivery includes several fields, timeouts are performed prior to each field. FIG. 2 illustrates the procedure of timeout.

The fifth step is the actual delivery of radiation.

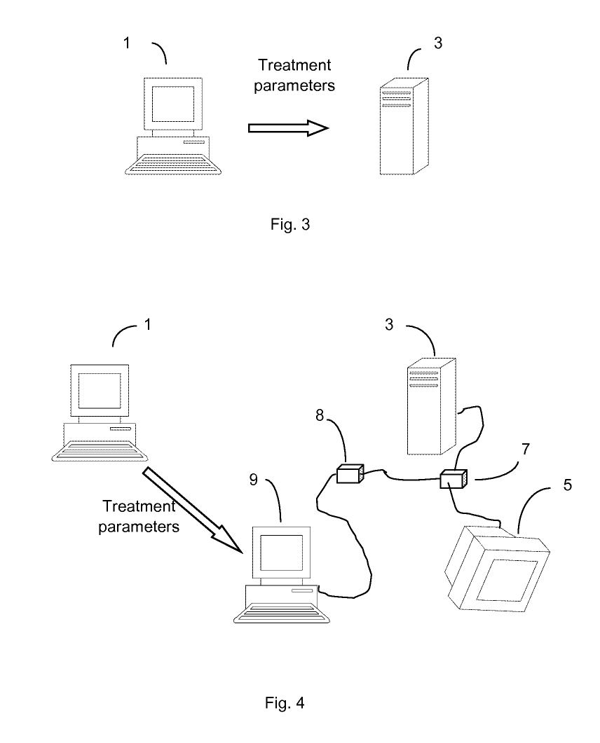

There are some variations of the dataflow shown in FIG. 1. Treatment planning data can be transferred to a treatment device directly, and either stored locally (e.g. Gammamed Plus from Varain Medical Systems) or transferred every time the patient receives the treatment (e.g. Tomotherapy or GammaMed Plus). Such flow of the data is shown in FIG. 3. However, the change of the workflow does not eliminate the need for the timeout step shown in FIG. 2.

There are three major disadvantages of the described timeout procedure. The first disadvantage is the time it takes to perform the timeout. Another disadvantage is that some discrepancies are not caught during the timeout. Unfortunately, it's difficult to find open source information on the latter point, because such information is, typically, not advertised by the hospital or the authorities. As some elucidation of the latter point we would like to mention that therapists are working under significant stress. Stress comes from time pressure to keep up on the busy treatment device. It also comes from the very fact that therapists often work with the terminally ill patients. The third disadvantage is the need to have two people for the timeout. This disadvantage makes radiation treatment by a single therapist inconvenient and awkward.

Thus, some kind of automation of the verification of treatment parameters is necessary. Nobody addressed, or, perhaps, even recognized, the problem before. The closest prior art is the use of the bar code for patient identification (U.S. Pat. No. 4,857,716 to Gombrich et al (1989) or U.S. Pat. No. 6,824,052 to Walsh (2004)).

### SUMMARY

We propose a method and apparatus for the automated verification of treatment field parameters (timeout) prior to radiation delivery to a patient. In one of the embodiments, video signal going to the monitor of the linac is captured using video splitter and VGA2USB converter. Optical character recognition is applied to the captured image, which contains actual treatment parameters. After that, the information extracted (the actual treatment parameters shown on the linac's screen) is compared with these parameters from the Treatment Planning System. The proposed procedure automates and mimics exactly the currently accepted manual procedure. The currently accepted manual procedure consists of manual comparison of the parameters on the linac screen with the parameters on the printout from the treatment planning system. However, the nature of our invention is to perform this procedure automatically, using computer vision tools.

### DRAWINGS

### Figures

FIG. 1. The diagram of the data flow from a treatment planning system to a radiation delivery device.

FIG. 2 The diagram of the verification of the treatment parameters, or a timeout procedure.

FIG. 3. The diagram of the data flow from a treatment planning system to a radiation delivery device when record-and-verify system is not used.

FIG. 4. First embodiment of automated timeout workflow and device.

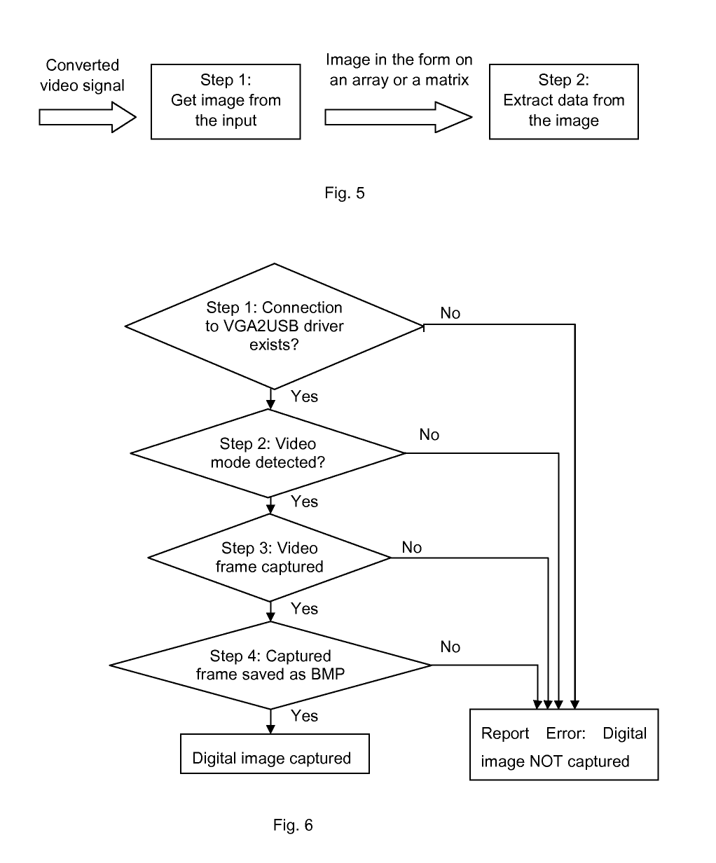

FIG. 5. Two major steps in extraction of necessary data from the signal from video to USB converter.

FIG. 6. Flow chart of the conversion of the USB signal to an image in the form of an array or matrix.

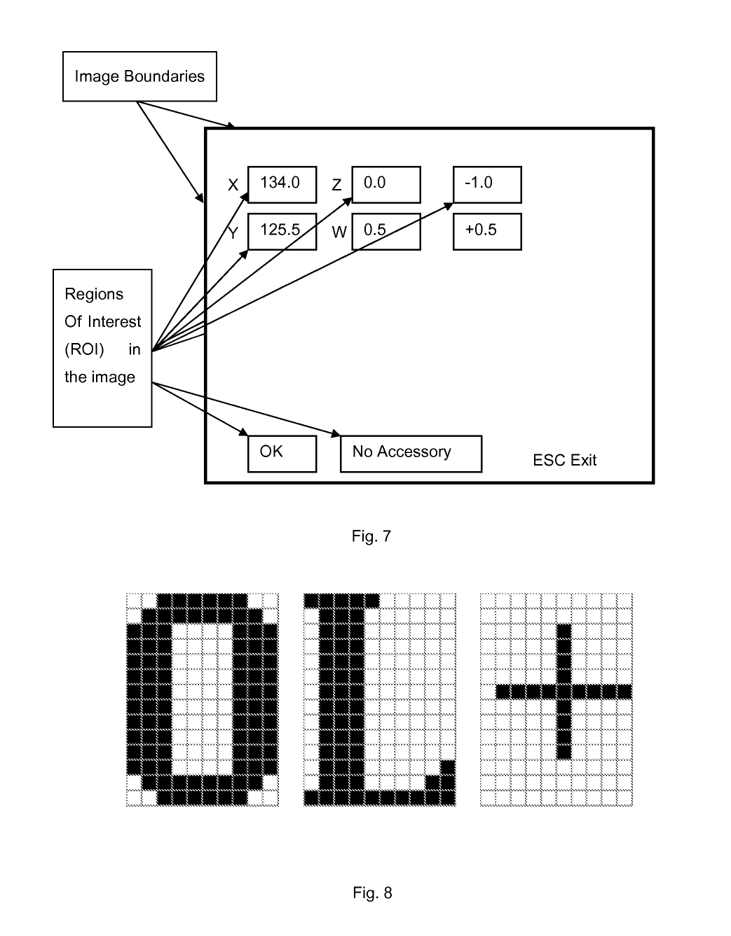

FIG. 7. An illustration of Regions of Interest (ROI) in a captured image of treatment parameters.

FIG. 8. Example symbol templates for symbols ‘0’, ‘L’, and ‘+’.

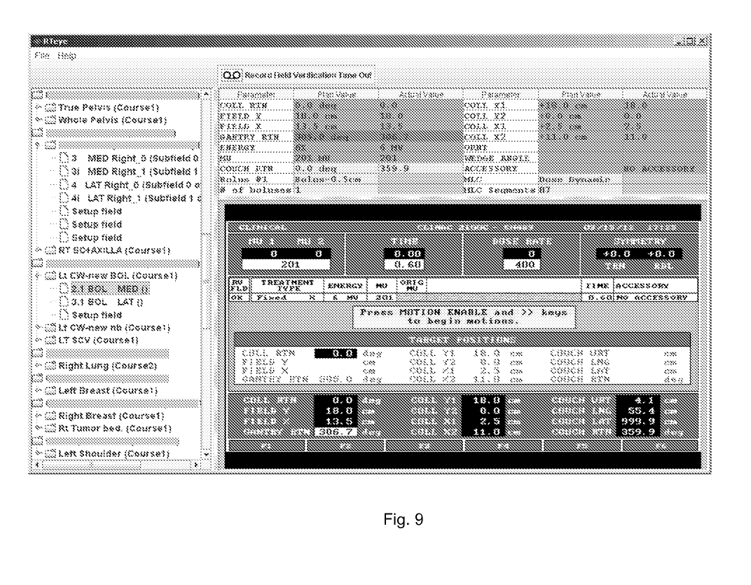

FIG. 9. Screen shot of the user interface in our implementation (of RTeye program).

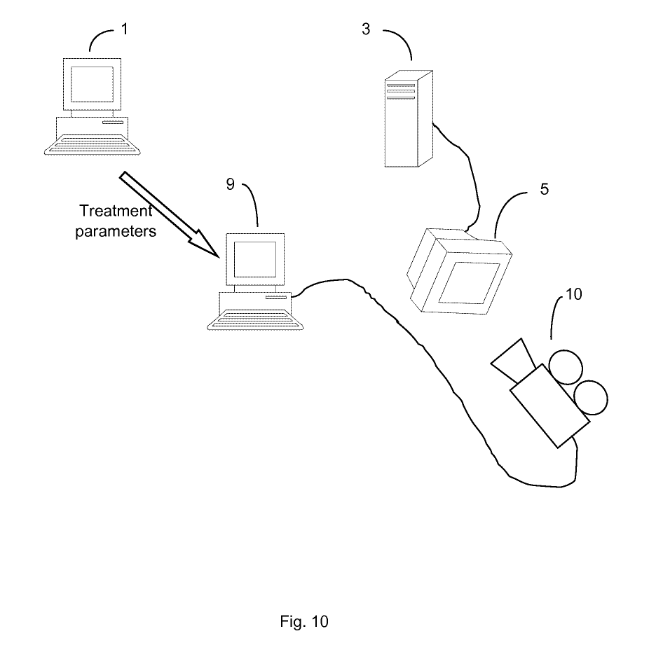

FIG. 10. Scheme of the third embodiment.

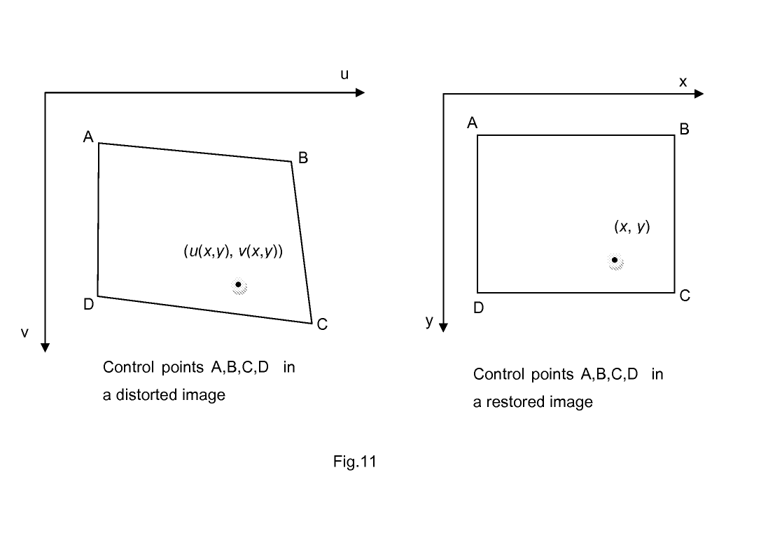

FIG. 11. Diagram showing the distorted monitor image and the restored image.

### DRAWINGS

### Reference numerals

1 treatment planning system
2 record-and-verify system
3 control system of radiation delivery device
4 treatment parameters from treatment planning system in the form of manual write up, printout or electronic printout
5 human interface device, e.g. monitor or printout
6 radiation technologist (therapist)
7 video signal splitter
8 video to USB converter
9 computer used to perform automatic timeout
10 device capable of capturing images, e.g. camcorder

### DETAILED DESCRIPTION OF THE INVENTION

A computer vision approach is proposed to replace a manual timeout procedure performed by the radiation therapists. Nobody proposed this approach previously. The idea is to digitize loaded treatment parameters directly from the screen of treatment device. Then these parameters are compared to the parameters extracted from the planning report prepared during planning stage. Specific implementations of the idea are presented below. The implementation described in the ‘First embodiment’ subsection has been built for one linac vendor and two vendors of the treatment planning system. As a part of the implementation, the program named RTeye has been developed. The implementation (including the program) is being evaluated for the clinical use in two clinics in the United States: Health Quest, Poughkeepsie, N.Y. and Steward Health, Methuen, Mass.

### First Embodiment

### Overview of the Device and Input to the Software From the User:

FIG. 4 shows the physical arrangement of the device for the verification of treatment parameters. The procedure during the timeout (the fourth step in the Background of the invention section) is as follows: in the automatic timeout software, radiation therapists select the patient to be treated, the treatment plan, and the treatment field, if the latter is applicable. In our implementation (in RTeye program we developed), selection is made using standard treeview control.

Here is what's happening on the software and workflow levels:

Treatment Parameters From the treatment Planning System:

After treatment plan is approved, i.e. at step two described in the Background of invention section, the treatment data is also stored for an electronic timeout. Data may be stored in some accessible network location or locally at the computer performing the timeout. Data is stored in the PDF or PostScript (PS) formats, usually easily generated by TPS. For more complicated situations other formats, or multistep conversion may be necessary.

In our implementation for Eclipse TPS (Varian Medical Systems, Palo Alto, Calif.), we use PDF printout. PDF is generated using common free PDF printers such as PrimoPDF (Nitro PDF, San Francisco, Calif.) or CutePDF (Acro Software Inc., Haymarket, Va.). Open source Java library available at http://pdfbox.apache.org/download.html was used for converting PDF files to text. The library function can be used in a Java application (as in our implementation) or as an operating system script can be run from the C++ or another program.

In our implementation for Pinnacle TPS (Philips Healthcare, Andover, Mass.), we use a PS printout.

### Treatment Parameters From the Treatment Device:

Signal coming from the controlling computer to human interface device, in this case a monitor, is intercepted using video signal splitter 7. While we expect that any video signal splitter would work, in our implementation we use ‘2-Way SVGA VGA splitter Amplifier Multiplier 400 MHz’ available through amazon.com. Then, the video signal is transferred to a computer for performing an automated timeout. While there are many approaches to perform this step, the easiest is, perhaps, to use a commercially available VGA (the format of the video signal from the control center of the radiation therapy device) to USB (universal serial bus—format suitable to serve as an input to most computers) converter, e.g. VGA2USB from Epiphan Systems, Inc. As is seen in FIG. 5, extracting treatment device data from the USB input is two-step process. First, data from the USB is converted to a more convenient representation of an image, such as array or matrix of pixels. Then, the treatment parameters are extracted from the image.

The flowchart of the image extraction from the USB input is shown in FIG. 6. A VGA2USB device is supplied along with Software Development Toolkit, including Dynamically-Link Libraries and Application Programming Interface definitions, allowing the image and video capture capabilities to be used from another program, written, for example in C++ or Java. Our automated timeout program (RTeye) utilizes these capabilities to capture digital images of treatment parameters from a radiation delivery device. The major steps of successful digital image capturing process are: verification that (1) a VGA2USB driver is running, (2) VGA2USB device is connected and has valid video parameters, (3) the video output frame can be successfully captured, and (4) the captured frame can be saved as a temporary file in Bitmap Image File (BMP). If one of the above-mentioned steps fails, an error with corresponding diagnostics can be reported by the program. It should be noted that at step 4 other image formats can be used for storing the captured image, or, alternatively, this step can be skipped, if it is possible to access digital image pixels of the captured frame from memory.

Our program is capable of extracting data from Varian linacs 600, 2100, iX and Trilogy (Varian Medical Systems, Palo Alto, Calif.). Explanation of the extraction of treatment data requires introduction of certain terms and concepts.

The data presented by the control system of radiation delivery device at the monitor are either numbers (e.g. position, angle or treatment time) or certain words or expressions (e.g. treatment type or accessories attached). Position of these numbers or words typically does not vary with time, which greatly simplifies the extraction of the treatment parameters from the captured image.

For each type of vendor video output we define (as we implement a program) a set of necessary parameters Pᵢ to be read from the image. Here i is the parameter index, an integer in the range 1 . . . N, and N is the number of treatment parameters to be verified during the procedure of automated timeout.

For the video output we assume some standard size of the captured image. Given that the captured treatment parameters image is of standard size, we define a set of rectangular regions on the image, called Regions Of Interest (ROI), so that each treatment parameter P, corresponds to a ROIᵢ on the image of standard size. Each ROI can be described by the coordinates of its two diagonal corners in the image, and the background and foreground colors inside ROI. Location and possible contents of ROIs are schematically presented in FIGS. 7 and 8. While our example implementation was focused on Varain linacs, we expect that our approach should work for any vendor after appropriate modifications.

The process of treatment parameters extraction from a captured image can be outlined as follows:
1) Detect and locate boundaries in the captured image.
2) Rescale the image to a standard size.
3) For each ROI in the image, convert ROI image region into a binary image form (background pixels set to 0, foreground pixels set to 1).
4) For each ROI image region in binary form, find a set of connected clusters of foreground pixels, with each connected cluster becoming a candidate symbol in the ROI.

5) For each candidate symbol in a ROI, find the best matching template among the templates of possible symbols in this ROI, assigning a dissimilarity score between each candidate symbol and the best matching symbol template. It should be noted that for some ROI, there might be no candidate symbols, i.e. the value of the parameter in this ROI is actually empty. 6) For each ROI, if a dissimilarity score of each candidate symbol is less than some threshold T, reconstruct the value of the parameter in the ROI by arranging the symbols of best matching templates in the same left-to-right order as the candidate symbols in the ROI. If a dissimilarity score in a ROI is above the threshold T, mark the captured value of the parameter represented by the ROI as ‘unrecognized’.

Below these steps are described in the greater details.

(1, 2) The boundaries of a captured image can be detected with the help of Prewitt edge detection operation (http://en.wikipedia.org/wiki/Prewitt operator), based on vertical and horizontal image gradients. If the boundaries found in the image are located at Xₘᵢₙ, Xₘₐₓ (horizontal boundaries) and Yₘᵢₙ, Yₘₐₓ (vertical boundaries) and in the standard image the boundaries are located at xₘᵢₙ, xₘₐₓ, and yₘᵢₙ, yₘₐₓ, each pixel value Iₛₜ(x, y) at coordinates x, y of the rescaled image is found in the following manner:

For x=xₘᵢₙ to xₘₐₓ
For y=yₘᵢₙ to yₘₐₓ

Iₛₜ(x,y)=I_{orig}[Round(Xₘᵢₙ+(x−xₘᵢₙ)/(xₘₐₓ−xₘᵢₙ)*(Xₘₐₓ−Xₘᵢₙ)),

Round(Yₘᵢₙ+(y−yₘᵢₙ)/(yₘₐₓ−yₘᵢₙ)* (Yₘₐₓ−Yₘᵢₙ))]

This method is an affine transform of the region in between the boundaries with the nearest-neighbor mapping.

(3) For each ROI, colors of the foreground and background are known. The components of the foreground color are denoted as C_{f}=(r_{f}, g_{f}, b_{f}), and the components for the background color are C_{b}=(r_{b}, g_{b}, b_{b}). The rule of making pixel p inside ROI as belonging to the foreground or background is as follows: Assume the color components of pixel p are Cₚ=(rₚ, gₚ, bₚ), where rₚ, gₚ, bₚ are red green and blue components usually used to represent color on the monitory, then the pixel p is marked a foreground if Distance(Cₚ, C_{f})<=Distance(Cₚ, C_{b}), otherwise, the pixel is marked as a background. The function Distance(,) can be defined as Euclidian distance between the color components. Distance(C_{b}, C_{f})=sqrt((r_{b}−r_{f})²+(g_{b}−g_{f})²+(b_{b}−b_{f})²), where sqrt denotes a square root. Each pixel inside its ROI is marked as foreground (1 in the binary image) or background pixel (0 in the binary image).

(4) For each binary image representation of ROI, find the 4-connected foreground components and mark them as symbol candidate symbols using the standard method, called Connected Component Labeling (http://en.wikipedia.org/wiki/Connected Component Labeling), where 4-connected neighbors are defined as in the following link: http://en.wikipedia.org/wiki/Pixel_connectivity) (5) A method of comparison of candidate symbols to a template and finding the best matching template is based on computing the sum of absolute differences (http://en.wikipedia.org/wiki/Sum of absolute differences) between the candidate symbol and a template for all possible positions of the template relative to the candidate symbol. Thus, the dissimilarity score between a template t, and a candidate symbol cⱼ is defined as:

$$S ( {t}_{i} , {c}_{j} ) = {min}_{{all relative}_{{positions of}_{template {t}_{i}}}} [ \begin{matrix}{\sum }_{{all pixel}_{{coordinate x , y i n}_{template {t}_{i}}}} \vert {t}_{i} ( x , y ) - {c}_{j} ( x , y ) \vert + \\ \{ \begin{matrix}number of foreground pixels \\ in {c}_{j} not overlapping with {t}_{i}\end{matrix} \}\end{matrix} ] ,$$

where: tᵢ (x, y) is the binary value of the pixel with coordinates (x, y) in the temple tᵢ, and cⱼ(x, y) is the binary value of the pixel with coordinates (x, y) in the candidate symbol cⱼ.

The best matching template to the candidate symbol cⱼ is found as

t_{best}(cⱼ)=argmin_{over all t}_{ᵢ}S(tᵢ, cⱼ),

and the symbol representing the test matching symbol for each candidate is considered the actual candidate symbol value.

The symbol templates are constructed from the actual captured images for all possible letters, digits and other symbols present in different ROI of the capture image. The example template for symbols ‘0’, ‘L’, and ‘+’ are provided in FIG. 8. Each template consists of the set of pixels belonging to the image of the corresponding symbol and surrounding it; and each foreground pixel (black) is coded with the pixel value of 1, and each background pixel (white) is coded with the pixel value of 0.

(6) In each ROI, all the symbols corresponding to the best matching candidate symbols in the ROI image are arranged in left-to-right order and the resulting set of symbols is concatenated to become the found value of the parameter in each ROI. Thus, a ROIᵢ would have a corresponding found value VPᵢ for the treatment parameter in ROIᵢ. The set of VPᵢ, for all possible values of index i is the output of the recognition program.

The output of our recognition program, namely, VPᵢ for each i are compared with the data from treatment planning system described in first paragraph of this section. A “pass” of the timeout does not necessarily mean the exact match as numbers may have certain tolerances and different accessories may be actually identical. The pass or fail criterion and parameters tolerances are established by the institution utilizing the timeout device.

### User Output

The result is conveyed to the user (one of the therapists) as pass, or fail, with the indication of the failed parameter(s). In our opinion, the most convenient way of the information output is in the table-like arrangement displayed on the computer monitor, when first column corresponds to parameter from TPS and second column to parameter extracted from treatment device. In our implementation we use two tables side by side, an arrangement more suitable to landscape screen orientation. The parameters that match the planned values (passed parameters) are highlighted by green and the parameters that fail the verification are highlighted by red. Those treatment parameters that are not available on the screen of treatment device, but still require verification, are highlighted in yellow.

In addition to alerting the user of parameters mismatch (using red color), our implementation also has a low level alert (indicated by yellow color) of mismatch which is within the configurable tolerance boundaries. This feature can come handy both clinically and for the testing of the program.

FIG. 9 shows a screen capture of the interface of our RTeye program. In this case: collimator rotation is outside 1° tolerance and indicated by red. Gantry rotation and one of the jaw positions do not match exactly, but are still within tolerance. They are highlighted with yellow color. The program also alerts that there is bolus and dynamic leaf motion. Patient names are obfuscated after the screen capture to protect patients' identities (New York state requirement).

Our program also has the capability of documenting the fact that the timeout procedure has been performed, by storing the timestamp of the timeout, along with all treatment parameters, in a file.

### Second Embodiment

This is similar to the first embodiment, except treatment planning data is extracted in the format, which does not directly contains text, i.e. treatment parameters are printed as images. In this case we need to use optical character recognition to read the printout from TPS. We can also read plan printout from the screen of the record-and-verify system or directly from the record-and-verify system's database, where plan printouts are, typically, saved as images, rather than in the format containing actual text.

### Third Embodiment

For the situation when the use of the splitter 7 in FIG. 4 is not possible, the workaround presented in FIG. 10 can be used. In this embodiment imaging device, e.g. video camera, 10, faces the human interface device, e.g. monitor, in the case monitor and transfers image to the computer 9 used to perform timeout. While data processing is similar to the first embodiment, image processing step presented in FIG. 6 requires some modifications. Image of the monitor would be distorted due to position of the camera, curvature of the monitor, imperfections of the camera lens, external light sources and so on. To partially mitigate the effect of distortions and reconstruct the image on the monitor, automated geometric correction, based on control points in the image and bilinear digital image transformation (resampling), should be used to reconstruct the part of the image containing all the ROIs, as defined in the previous embodiments. Control points are the points in the original image on the display, which can be automatically recognized in the image from the camera, such as corners of the display, corners of large clusters, and distinct line intersections. As an option, control points can be introduced into the image artificially, for example, by attaching a number of round (or some other distinctly shaped or colored) stickers to the corners of the monitor.

To illustrate the process of automated geometric correction, let's assume the image contains some control points A, B, C, and D, which, in undistorted image represent the corners of some rectangle. The sides of the rectangle ABCD are parallel to the sides of the undistorted image, the coordinates of the rectangle corners are known, and have the following properties:
X_{A}=X_{D}; X_{B}=X_{C}; Y_{A}=Y_{B}; Y_{D}=Y_{C};

When the corresponding control points are found in the distorted image, as shown in the left image of the FIG. 11, these control points would have the corresponding coordinates in the distorted image:
A at (U_{A}, V_{A}), B at (U_{B}, V_{B}), C at (U_{C}, V_{C}), D at (U_{D}, V_{D}).

Given the coordinates of the control points in the undistorted image, and the coordinates of these in the distorted image, a bilinear image transformation (similar to described at this link: http://en.wikipedia.org/wiki/Bilinear interpolation), is applied to find out the pixel values inside the rectangle ABCD in the restored image. Let's denote as Iᵣ(x,y) the pixel value (or color components) at coordinates (x, y) in the restored image, where pixel (x, y) lays inside the rectangle of the control points ABCD; let's denote as I_{d}(u, v) the pixel value at coordinates (u, v) in the distorted image. In this case, the color components of the pixels inside the rectangle ABCD of the restored image are found using the following formula:

(x, y)=I_{d}(u(x, y), v(x, y)),

where u(x, y) and v(x, y) are:

$$u ( x , y ) = \frac{\begin{matrix}( y - {Y}_{A} ) [ {U}_{A} ( x - {X}_{A} ) + {U}_{B} ( {X}_{B} - x ) ] + \\ ( {Y}_{D} - y ) [ {U}_{C} ( {X}_{C} - x ) + {U}_{D} ( x - {X}_{D} ) ]\end{matrix}}{( {X}_{B} - {X}_{A} ) ( {Y}_{D} - {Y}_{A} )}$$

$$and$$

$$v ( x , y ) = \frac{\begin{matrix}( x - {X}_{A} ) [ {V}_{A} ( y - {Y}_{A} ) + {V}_{D} ( {Y}_{D} - y ) ] + \\ ( {X}_{B} - x ) [ {V}_{B} ( {Y}_{D} - y ) + {V}_{C} ( y - {Y}_{D} ) ]\end{matrix}}{( {X}_{B} - {X}_{A} ) ( {Y}_{D} - {Y}_{A} )}$$

The right image in FIG. 11 shows the region ABCD in the restored image, whereas the both images in FIG. 11 show the mapping between the distorted image (left) and the restored image (right).

It should be noted that:
The described example can be generalized for the cases when the regions of control points are not necessarily rectangular.
In restoring the image, it is desirable to have as many different regions of control points as possible, and the regions of control points should be relatively small as compared to the size of the entire image.
The size and the number of regions of control points for output images should be determined individually for each provider, as a trade-off of computational and algorithmic complexity to find the control points in the distorted image on one hand, and the quality of the resulting restored image on the other hand.

### Ramifications and Scope

A is seen from the above, an automatic timeout provides a faster and more reliable way for the verification of the treatment parameters.

Although the description above contains much specificity, these should not be construed as limiting the scope of the embodiments, but as merely providing illustrations of some of the presently preferred embodiments.

Certain additions are possible to improve the efficiency of the automatic timeout procedure. For example, the name of the patient under treatment and other identification information, rather than being chosen manually by the therapist, can be extracted from the database of the record-and-verify system, read from the screen of the record-and-verify system or read using patient ID scanner.

Human interface device 5 in FIG. 2 does not have to be a monitor. For example, in can be a printer, so that printout sent, say, to the USB port would be captured and analyzed in real time.

Another possible workflow, which would not involve treatment planning system directly, is to perform a timeout prior to the first treatment fraction manually, and to record the image for the monitor 5 (FIG. 4) using our timeout device. Then, during the following treatment fractions, this recorded image can be used instead of the treatment planning data for the comparison in the subsequent timeouts. In this situation two images can be compared directly, as images, without the extraction of the necessary information.

In some cases, the treatment parameters are not confined to a single monitor or a single controlling computer. In this case timeout device uses input intercepted from two or more monitors, or other human interface devices.

Certain treatment parameters are presented in the graphical form, e.g. as some geometric shape. As an example, the shape of multileaf collimator (MLC—a special set of leafs designed to create comprehensive shape of the radiation beam for external beam treatment) is shown on some radiation producing devices. While radiation therapists may review this information, this review is only qualitative. Computer based timeout device may get more quantitative information and compare it with the treatment planning system.

Certain treatment parameters are monitored by the therapist during the treatment delivery. For example, some treatment delivery scenarios involve continuous or stop-and-move motion of MLCs. Typically, a radiation therapist monitors that MLC actually moves during the treatment. A computer based timeout device may actually monitor that leafs, or something else changing dynamically, e.g. dose rate or collimating jaws, do perform a correct motion.

The computer performing the automatic timeout does not have to be a separate entity. For example, the software used to perform the timeout can be installed on the same computer as the treatment planning system, or on the record-and-verify system, or on some other computer, which already exists in clinical settings. It would be the user's responsibility, though, to make sure that such an installation does not violate vendor's requirements.

### Advantages

The following are some of the advantages of the automatic timeout over a manual timeout
(a) Automatic timeout is not prone to human error;
(b) Automatic timeout in many cases can be performed faster than the manual timeout;
(c) The therapist can focus on their direct duty, working with the patient, prior and during the treatment;
(d) Automatic timeout procedure allows a single therapist to perform the verification of treatment parameters.

1. A method of verification by automatic means of some or all treatment parameters for a patient before, during and/or after treatment delivery of a radiation treatment device

2. A method according to claim 1 wherein said means comprised of a computer

3. A method according to claim 2 comprising of converting into an image or images the output or outputs from control system or systems of radiation delivery device to human interface device or devices

4. A method according to claim 3 wherein the conversion is preceded by the use of the splitter for the signal from the control system or systems to human interface device or devices

5. A method according to claim 3 wherein the conversion is preceded by the use of video acquisition device capturing the image or images on the human interface device or devices

6. A method according to claim 3 further comprising of comparison of this image or images to the image of images acquired during first treatment (fraction)

7. A method according to claim 3 further comprising of extracting data from this image or these images

8. A method according to claim 7 further comprising of comparing these data to the data from the treatment planning system

9. A method according to claim 8 wherein data from the treatment planning system is extracted via intermediate file or files

10. A method according to claim 9 wherein intermediate files are electronic printouts

11. A method according to claim 9 wherein extraction involves optical character recognition

12. A method according to claim 9 wherein intermediate files are stored in the record-and-verify system

13. A method according to claim 8 wherein data from the treatment planning system is extracted from treatment planning system database

14. A method according to claim 8 wherein data from the treatment planning system is extracted using image or images on the screen

15. A method according to claim 14 wherein image or images are shown by record-and-verify system

16. A method according to claim 14 wherein image or images are shown by treatment planning system

17. A method according to claim 14 wherein extraction involves optical character recognition

18. A method according to claim 7 further comprising of comparing these data to the data extracted from the images acquired during first treatment (fraction)

19. A method according to claim 2 wherein the computer is one of the computers already present in clinical settings

20. A method according to claim 2 wherein identification of the patient being treated is read from the database of record-and-verify system

21. A method according to claim 2 wherein identification of the patient being treated is read using patient ID scanner

22. A method according to claim 2 wherein identification of the patient being treated is taken from a pre-created schedule

23. A method according to claim 2 wherein identification of the patient being treated is taken from the human interface device of record-and-verify system
